(19) United States
(12) Patent Application Publication
(10) Pub. No.: US 2018/0366142 A1
(43) Pub. Date: Dec. 20, 2018

(54) PREDICTIVE NOTIFICATION OF PERSONALITY SHIFTS FOR MENTAL ILLNESS MANAGEMENT

(71) Applicant: INTERNATIONAL BUSINESS MACHINES CORPORATION, Armonk, NY (US)

(72) Inventors: Maryam ASHOORI, WHITE PLAINS, NY (US); Benjamin D. BRIGGS, WATERFORD, NY (US); Lawrence A. CLEVENGER, DUTCHESS, NY (US); Leigh Anne H. CLEVENGER, RHINEBECK, NY (US); Michael RIZZOLO, ALBANY, NY (US)

(21) Appl. No.: 15/804,333

(22) Filed: Nov. 6, 2017

Related U.S. Application Data
(63) Continuation of application No. 15/622,577, filed on Jun. 14, 2017.

(51) Int. Cl. G10L 25/63; A61B 5/16; G10L 15/26; G06F 19/00

(52) U.S. Cl. CPC G10L 25/63; A61B 5/165; G10L 15/26; G06F 19/3406; G06F 19/3437; G06F 19/3431

(57) ABSTRACT

Embodiments of the present invention are directed to a computer program product for generating a personality shift determination. The computer program product can include a computer readable storage medium having program instructions embodied therewith, wherein the instructions are executable by a processor to cause the processor to perform a method. The method can include receiving a real-time audio input. The method can also include generating a real-time personality trait identification. The method can also include generating a current trait classification for the real-time personality trait identification. The method can also include comparing the current trait classification to a historic rate classification. The method can also include generating a personality shift determination.

### DOMESTIC AND/OR FOREIGN PRIORITY

This application is a continuation of U.S. application Ser. No. 15/622,577, titled “Predictive Notification of Personality Shifts for Mental Illness Management” filed Jun. 14, 2017, the contents of which are incorporated by reference herein in its entirety.

### BACKGROUND

The present invention generally relates to mental illness management. More specifically, the present invention relates to predictive notification of personality shifts for mental illness management.

A number of mental illnesses and ailments are associated with personality shifts. For example, schizophrenia, multiple personality disorders, and autism can be associated with often abrupt personality shifts that can arise with little to no warning to family and care providers.

In some cases, such personality shifts can adversely impact not only the patient but also caregivers and family members and can call for medical or behavioral intervention. For example, a schizophrenic patient can experience a sudden personality change that causes the patient to lose touch with reality and, for instance, can pose a risk of harm to himself and others surrounding him or her if the change is undetected or untreated. Similarly, for example, autistic patients can experience shifts in personality that can result in self-injurious behaviors. Advance notice of such personality shifts could allow caregivers and family members to prepare for adverse episodes, for example by enabling them to take proactive steps to ensure the safety of the patient and others around them.

### SUMMARY

Embodiments of the present invention are directed to a computer-implemented method for personality shift determination. A non-limiting example of the method includes receiving, by a processor, a real-time audio input including spoken words of a person. The method also includes generating, by the processor, a real-time personality trait identification based at least in part upon the real-time audio input. The method also includes generating, by the processor, a current trait classification for the real-time personality trait identification based at least in part upon the real-time audio input and a trait classification model. The method also includes comparing, by the processor, the current trait classification to a historic rate classification. The method also includes generating, by the processor, a personality shift determination based at least in part upon the comparison. Such embodiments can provide, for example, prediction of personality shifts for medical and psychological disorders without need for lengthy clinical evaluations.

Embodiments of the present invention are directed to a computer program product for personality shift determination. The computer program product can include a computer readable storage medium having program instructions embodied therewith, wherein the instructions are executable by a processor to cause the processor to perform a method. A non-limiting example of the method includes receiving a real-time audio input. The method can also include generating a real-time personality trait identification based at least in part upon the real-time audio input. The method can also include generating a current trait classification for the real-time personality trait identification based at least in part upon the real-time audio input and a trait classification model. The method can also include comparing the current trait classification to a historic rate classification. The method can also include generating a personality shift determination based at least in part upon the comparison. Such embodiments of the invention can, for example, provide analysis of the efficacy of treatments for conditions involving personality shifts.

Embodiments of the present invention are directed to a processing system for personality shift determination. The processing system can include a processor in communication with one or more types of memory. A non-limiting example of operating the processing system includes a processor configured to receive a real-time audio input including spoken words of a person. The processor is also configured to identify a real-time personality trait based at least in part upon the real-time audio input. The processor is also configured to generate a current trait classification for the real-time personality trait based at least in part upon the real-time audio input and a trait classification model. The processor is also configured to compare the current trait classification to a historic rate classification. The processor is also configured to generate a personality shift determination based at least in part upon the comparison.

Embodiments of the invention are directed to a system for notification of personality shifts. A non-limiting example of the system includes a microphone in communication with a speech to text module. The system can also include a personality trait extraction module in communication with the speech to text module. The system can also include a personality shift prediction module in communication with the personality trait extraction module. The system can also include a user interface in communication with the personality shift prediction module. Such embodiments can, for example provide early warning of personality shifts for medical and psychological disorders to enhance caregiving and treatment of such disorders.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a cloud computing environment according to one or more embodiments of the present invention;

FIG. 2 depicts abstraction model layers according to one or more embodiments of the present invention;

FIG. 3 depicts a computer system according to one or more embodiments of the present invention;

FIG. 4 depicts a schematic of a system for generating personality shift data according to one or more embodiments of the present invention;

FIG. 5 depicts a flow diagram illustrating a method for generating personality shift data according to one or more embodiments of the invention;

FIG. 6 depicts an aspect of an exemplary personality trait analysis according to one or more embodiments of the present invention; and

FIG. 7 depicts an aspect of an exemplary personality trait analysis according to one or more embodiments of the present invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the described embodiments, the various elements illustrated in the figures are provided with two or three digit reference numbers. With minor exceptions, the leftmost digit(s) of each reference number correspond to the figure in which its element is first illustrated.

### DETAILED DESCRIPTION

A number of medical and psychological conditions involve personality shifts. The phrase “personality shifts,” and variations thereof, are used in this detailed description to include abnormal personality and behavioral shifts associated with a medical or psychological condition, such as schizophrenia, multiple personality disorder, autism, Alzheimer's, dementia, anxiety disorders, posttraumatic stress disorder, bipolar disorder, and borderline personality disorders. Personality shifts can be associated with confusion, delirium, delusions, hallucinations, mood extremes, and disorganized or erratic speech or behavior.

Personality shifts that occur without warning can be challenging for health care providers and family members to attend to and mitigate and affect a significant number of individuals in the United States and world-wide.

Schizophrenia, for example, is a chronic and severe neurological brain disorder that affects about 1.1 percent of the population or approximately 3.5 million adults in the United States. An estimated 40 percent of individuals with the condition are untreated in any given year. Not only can schizophrenia be a devastating disorder for those afflicted, but it can also involve significant monetary expenditures for affected families and for society at large. In the year 2002, for instance, expenditures related to treatment and care of schizophrenia were estimated to at over $60 billion, including direct health care costs, inpatient and outpatient costs, and costs pertaining to medications and long-term care.

Personality change can be a key factor in recognizing schizophrenia and schizophrenic episodes. Initial changes in personality by those experiencing a schizophrenic episode can be subtle or minor and can in some cases go unnoticed. Over time, however, such shifts can become readily apparent to family, friends, classmates or co-workers.

Early detection of personality shifts can provide family members and health care providers with time to take proactive steps to prepare for an episode, for instance, by providing time to ensure the safety of the patient and those around them.

Conventional methods of monitoring personality shifts can be difficult and impracticable to employ on a day to day basis. For example, written or computerized diagnostic quizzes and assessments that involve analysis of user responses to automated questions can be used to assess personality traits. However, the time involved in administering the assessments alone, much less the time needed to analyze the results, render such methods ineffective at providing advance notice of a personality shift. Collective brain measurements, on the other hand, can provide timely information on brain activity but can be impractical or impossible to use on a day to day basis to the extent they involve intrusive, cumbersome, and costly equipment.

Turning now to an overview of the aspects of the invention, one or more embodiments of the invention address the above-described shortcomings of the prior art by using speech to text analysis and physical sensor analysis with machine learning to generate personality shift data that can be determinative or predictive of personality shifts for medical and psychological disorders. Embodiments of the invention can generate a determination of the efficacy of treatments for conditions involving personality shifts, for example by generating a notification identifying an increase or decrease in the rate or duration of personality shift events.

The above-described aspects of the invention address the shortcomings of the prior art by providing systems that collect and monitor a patient's speech and movement patterns to generate a notification of a personality shift event. In some embodiments of the invention, a personality shift event notification can be generated and output before the personality shift is observable to external individuals, such as health care providers and family members. Embodiments of the invention provide continuous or near-continuous monitoring of spoken words by an individual through the use of microphones. Embodiments of the invention can include identification and analysis of personality traits and generation of personality trait identifications and classifications. A machine learning module can analyze speech and/or movement of an individual to generate personality shift determinations and notifications. In some embodiments of the invention, when a positive personality shift is detected, the related personality trait and duration of the trait or event can be recorded and used to classify patterns of personality shifts in real-time and over extended periods of time to predict when a personality shift is likely to happen or to generate an output of a predicted frequency and duration of a personality shift. In some embodiments of the invention, historic patterns of personality shifts can be generated and used to design treatment plans for patients, identify potential causes triggering a personality change, or to evaluate the effectiveness of medications and prescribed treatments for the associated disorder.

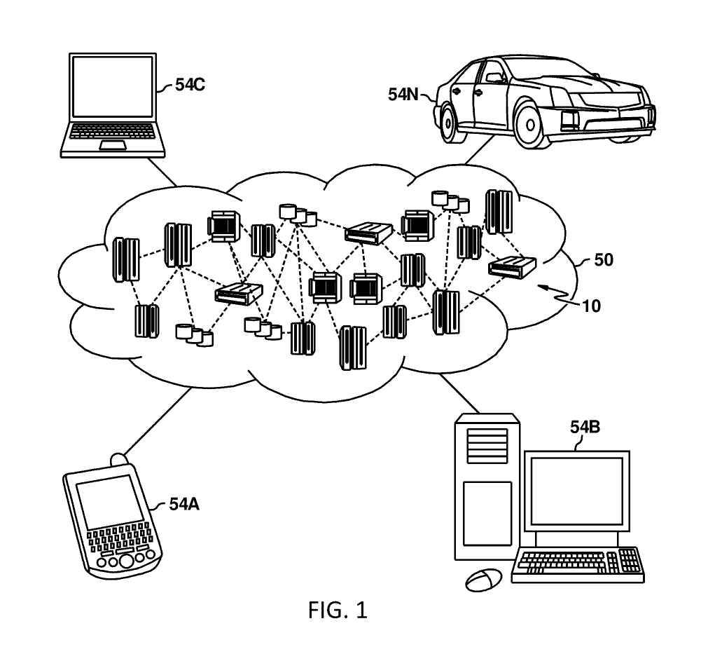

Turning now to a more detailed description of aspects of the present invention, referring now to FIG. 1, an illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N can communicate. Nodes 10 can communicate with one another. They can be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 1 are intended to be illustrative only and that computing nodes 10 and cloud computing environment 50 can communicate with any type of computerized device over any type of network and/or network addressable connection (e.g., using a web browser).

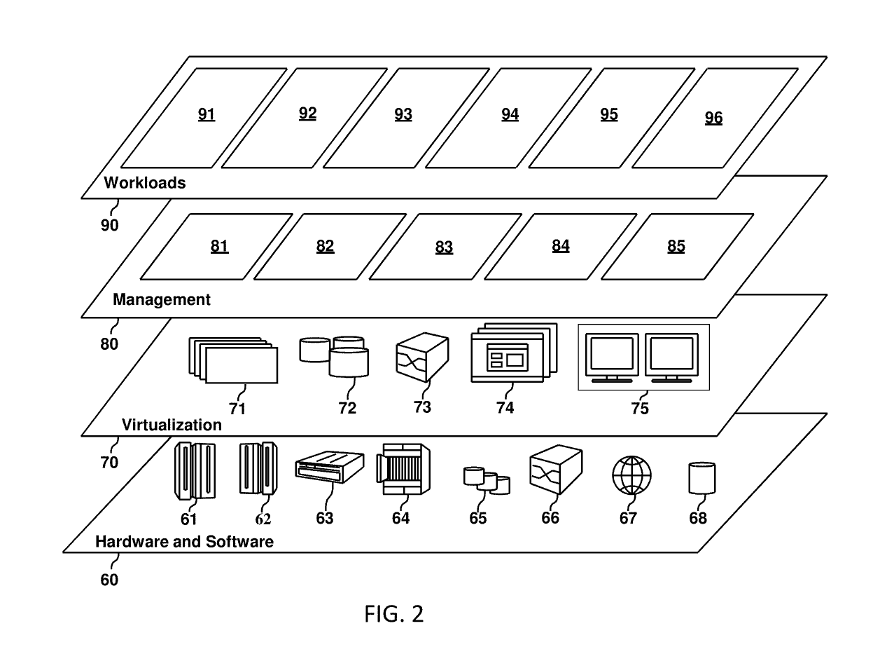

Referring now to FIG. 2, a set of functional abstraction layers provided by cloud computing environment 50 (FIG. 1) is shown. It should be understood in advance that the components, layers, and functions shown in FIG. 2 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities can be provided: virtual servers 71; virtual storage 72; virtual networks 73, including virtual private networks; virtual applications and operating systems 74; and virtual clients 75.

In one example, management layer 80 can provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources can include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment can be utilized. Examples of workloads and functions which can be provided from this layer include: mapping and navigation 91; software development and lifecycle management 92; virtual classroom education delivery 93; data analytics processing 94; transaction processing 95; and personality shift prediction 96.

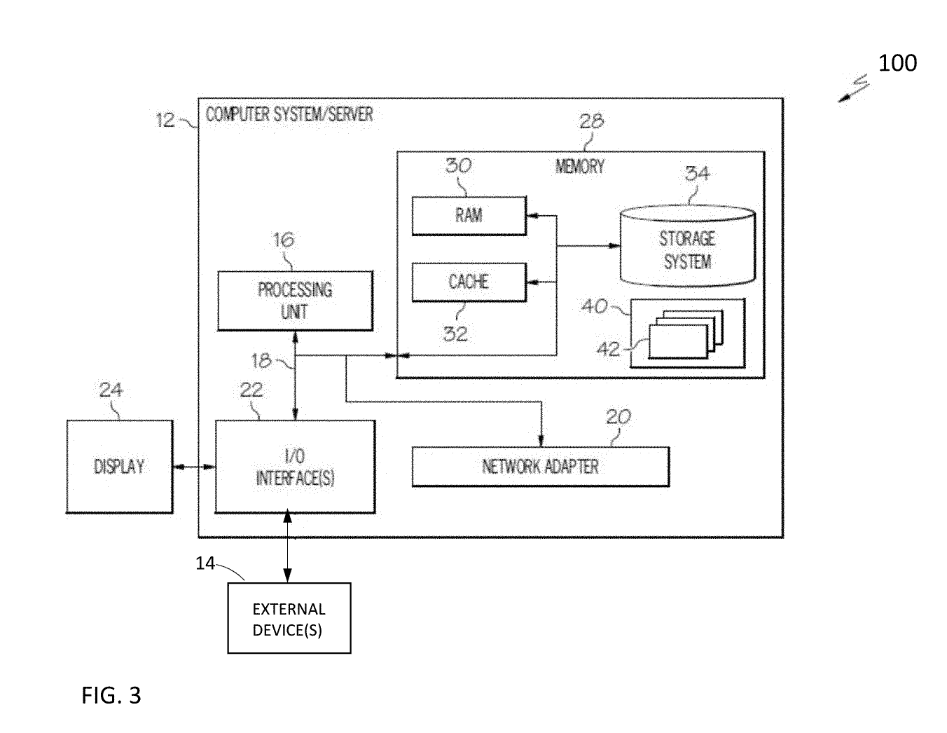

Referring now to FIG. 3, a schematic of a cloud computing node 100 included in a distributed cloud environment or cloud service network is shown according to a non-limiting embodiment. The cloud computing node 100 is only one example of a suitable cloud computing node and is not intended to suggest any limitation as to the scope of use or functionality of embodiments of the invention described herein. Regardless, cloud computing node 100 is capable of being implemented and/or performing any of the functionality set forth hereinabove.

In cloud computing node 100 there is a computer system/server 12, which is operational with numerous other general purpose or special purpose computing system environments or configurations. Examples of well-known computing systems, environments, and/or configurations that can be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 can be described in the general context of computer system-executable instructions, such as program modules, being executed by a computer system. Generally, program modules can include routines, programs, objects, components, logic, data structures, and so on that perform particular tasks or implement particular abstract data types. Computer system/server 12 can be practiced in distributed cloud computing environments where tasks are performed by remote processing devices that are linked through a communications network. In a distributed cloud computing environment, program modules can be located in both local and remote computer system storage media including memory storage devices.

As shown in FIG. 3, computer system/server 12 in cloud computing node 100 is shown in the form of a general-purpose computing device. The components of computer system/server 12 can include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media can be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 can further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 can include at least one program product having a set (e.g., at least one) of program modules that are configured to carry out the functions of embodiments of the invention.

Program/utility 40, having a set (at least one) of program modules 42, can be stored in memory 28 by way of example, and not limitation, as well as an operating system, one or more application programs, other program modules, and program data. Each of the operating system, one or more application programs, other program modules, and program data or some combination thereof, can include an implementation of a networking environment. Program modules 42 generally carry out the functions and/or methodologies of embodiments of the invention as described herein.

Computer system/server 12 can also communicate with one or more external devices 14 such as a keyboard, a pointing device, a display 24, etc., one or more devices that enable a user to interact with computer system/server 12, and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

Turning now to a more detailed discussion embodiments of the present invention, embodiments of the invention include systems and methods for predictive notification of personality shifts through continuously monitoring personality traits for a patient, such as a schizophrenic patient or other person undergoing observation or treatment for conditions that include personality shifts. Embodiments of the invention include microphones that can continuously receive spoken word input from the person. Personality traits can be identified based at least upon the spoken word. In some embodiments of the invention, a machine learning module, such as a personality shift prediction module, can look for shifts in personality. When a shift is detected, in some embodiments of the invention, the trait and the duration of the trait being observed is recorded. The machine learning module can classify patterns of personality shifts and over time, in some embodiments of the invention, can predict when a personality shift is about to occur. In some embodiments of the invention, the patterns can be used for designing a treatment plan for a person. In some embodiments of the invention, the patterns can be used to identify potential causes or triggers of a personality shift.

In some embodiments of the invention, physical movement of a person can be monitored, for instance along with monitoring of spoken word. Personality shifts, in some cases, can be accompanied by physical movements. Motions sensors, such as accelerometers and other wearable devices for sensing movement, can collect movement data. Some embodiments of the invention include processing and analysis of movement data, such as Fourier Transform analysis. For instance, an initial low frequency can indicate smooth movement. A subsequent high frequency can signal a change in a physical movement pattern. In some embodiments of the invention, repetitive physical movements can signal a personality shift.

In some embodiments of the invention, frequency and patterns of personality shifts can be used to evaluate the efficacy of medications and/or prescribed treatments.

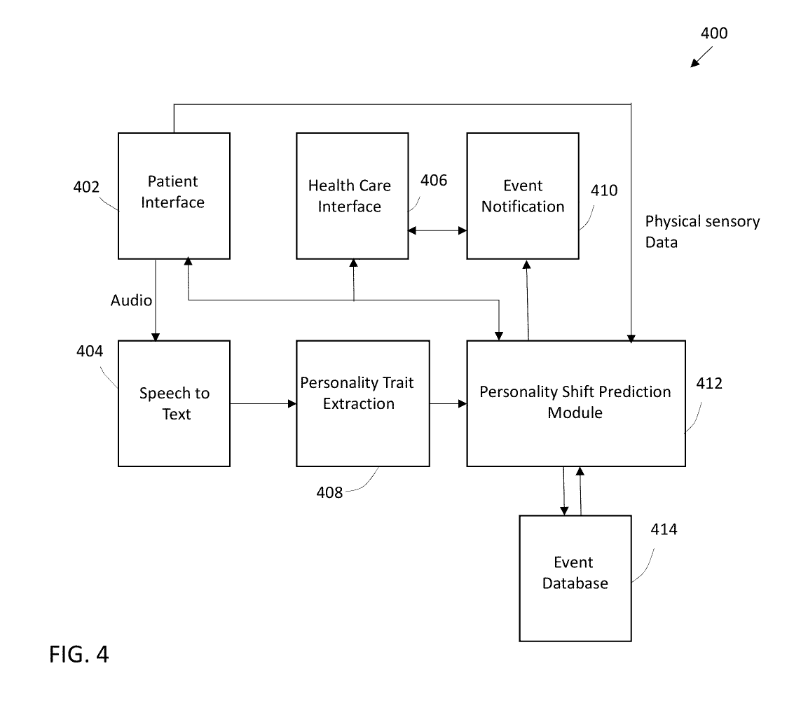

Referring now to FIG. 4, a block diagram illustrating an exemplary system 400 for notification of personality shifts is provided. The system 400 can include a speech to text module 404. The speech to text module 404 can include any system or service capable of converting spoken speech to text, such as WATSON™ Speech to Text or AMAZON ALEXA. The speech to text module 404 can receive an audio input from a patient interface 402. The system 400 can also include a personality trait extraction module 408. Personality extraction module 408 can include any system capable of extracting personality traits, such as Watson Personality Insight. The personality trait extraction module can receive input, including text input derived from patient speech, from the speech to text module 404. The personality trait extraction module 408 can communicate with a personality shift prediction module 412. The personality shift prediction module 412 can use machine learning to determine and/or predict a personality shift for the patient 412. The personality shift module 412 can also communicate with an event database 414 and a health care interface 406, such as a computer interface at a health care provider office or a smart phone or tablet of a nurse or local caregiver.

Communication between the system components can be wireless or wired communication and can include, for instance, Wi-Fi, cellular, Bluetooth, and/or RF communication.

Components of the system can be included in specialized or consumer wearable devices, including but not limited to smart phones, smart watches, wearable microphones, such as microphone necklaces, directional microphone necklaces, or wearable audio recorders, such as Instamic. In some embodiments of the invention, directional microphones are included in the system, for instance to aid in filtration of unwanted noises. In some embodiments of the invention, the system 400 can include speaker recognition systems to differentiate speech of the patient from other voices.

The event database 414 can include data, such as personality traits and corresponding event data, concerning the person and/or similarly situated persons. Similarly situated persons can include persons having the same or similar demographics and/or the same or similar medical or psychological diagnostics.

The personality shift prediction module 412 can also receive physical sensor data from the person 402. The physical sensory data can include, for example, motion data, including data derived or received from accelerometers, gyrometers, altimeters, global positioning devices, and the like and biophysical sensor data, such as heart rate, heartbeat, heart intensity, temperature, blood pressure, respiration rate, hormonal or blood sugar levels, and other data obtained or derived from wearable devices such as heart rate monitors, body temperature sensors, blood oxygen sensors, breathing rate sensors, breathing volume sensors or EDA (electro dermal activity) sensors.

In some embodiments of the invention, the personality shift prediction module can receive clinical information. Clinical information can be obtained automatically, for instance from electronic medical records, or through input from health care professionals, such as information keyed in from a clinician. Clinical information can include patient diagnosis, demographic data, such as age or gender, prescribed medications, prior diagnosis, medical events, and the like.

The personality shift prediction module 412 can correlate descriptors from text with personality traits, optionally along with volume or decibel level, physical movement patterns, and clinical information. Correlation can include, for example, application of multiple linear regression, partial least squares fit, Support Vector Machines, and random forest methods to the data.

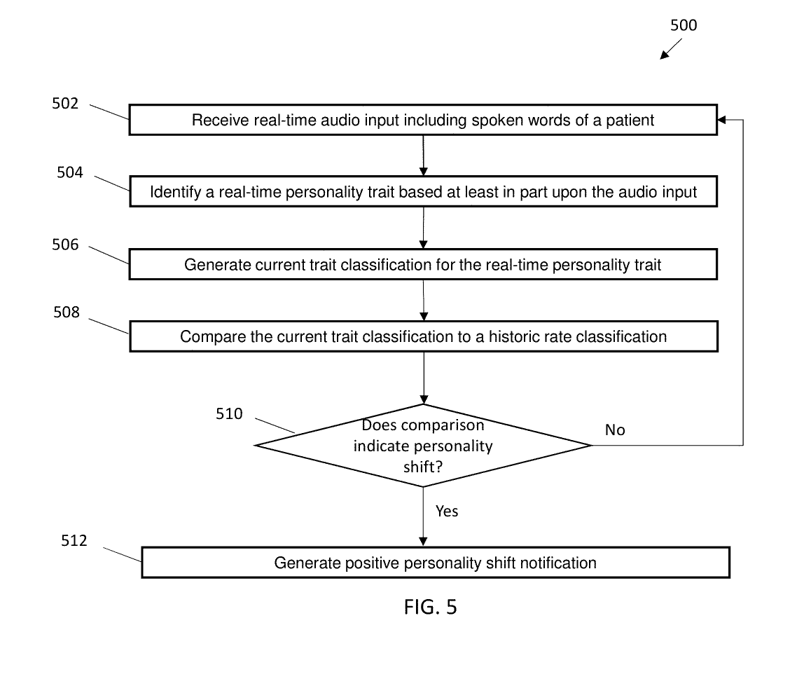

FIG. 5 depicts a flow diagram of an exemplary method of identifying personality shifts 500 according to one or more embodiments of the present invention. The method 500 can include, as shown at block 502, receiving a real time audio input including spoken words of a person. For example, as a person starts talking, the audio of every sentence can be collected from the microphone on a wearable device or smart watch. In some embodiments of the invention, transcription can be provided for a short period of time or for a minimum number of spoken words (such as 100 words). The method 500 can also include, a shown at block 504, identifying a real-time personality trait based at least in part upon the audio input. The method 500 can also include, as shown at block 506, generating a current trait classification for the real-time personality trait. The method 500 can also include, as shown at block 508, comparing the current trait classification to a historic rate classification. The historic rate classification can include historic data on personality shift behavior associated with shifts for the person in prior events or for persons with the same or similar medical or psychological conditions. The method 500 can include, as shown at decision block 510, determining whether the comparison indicates a personality shift. If the comparison does not indicate a personality shift, the method 500 can return to block 502. If the comparison indicates a personality shift, the method 500 can proceed to block 512 and can include generating a positive personality shift notification. Positive personality shift notification, as used herein, means a notification indicating a personality shift is likely to occur in the near future or is in progress and can reflect, in some embodiments of the invention, that a comparison exceeded a threshold value such as to render it statistically indicative of a personality shift.

In some embodiments of the invention, speech data can be supplemented by wearable sensor information on changes in physical movement patterns, which often accompany schizophrenic behavior and behavior associated with personality shifts in other medical or psychological conditions.

In some embodiments of the invention, a method includes saving a personality trait in a database. For example, if a personality shift is detected, the time/trait can be flagged and recorded as a personality shift event.

In some embodiments of the invention, family, caregivers, clinicians, and/or patients can be notified of a detected personality shift, for instance by a graphic illustration on a display, an audible notification through a microphone, or a haptic or other sensory notification, such as vibration or heating of a device.

In some embodiments of the invention, family, caregivers, clinicians, and/or patients can provide a response to a notification. For example, an individual can report a positive personality shift notification as a false event. In some embodiments of the invention, in response to a notification of a false event, a system can analyze the situation and modify personality shift analysis methods for future events.

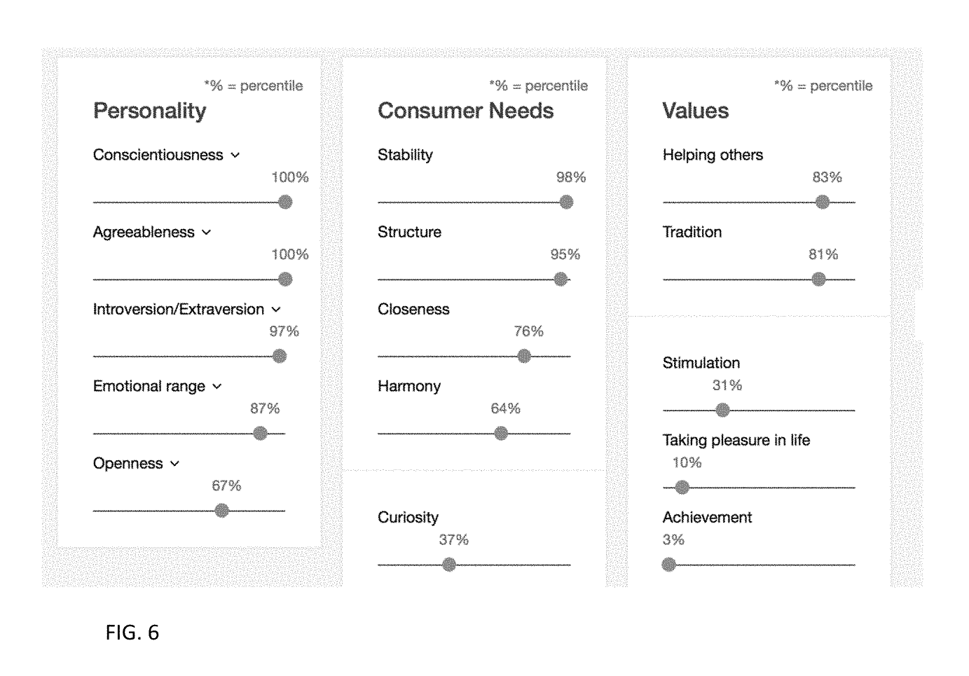

FIG. 6 depicts an aspect of an exemplary personality trait analysis according to one or more embodiments of the present invention. FIG. 6 shows an exemplary personality trait analysis indicative of positive speech, such as speech indicating that a psychologically or medically abnormal personality is absent. The personality trait analysis can include a trait identification with a trait classification. Traits that can be identified can include, for example, personality traits, such as conscientiousness, agreeableness, introversion/extraversion, emotional range, and openness; consumer needs, such as stability, structure, closeness, harmony, curiosity, excitement, love, and practicality; values, such as helping others, tradition, stimulation, taking pleasure in life, achievement, and tradition. The traits can be classified relative to the person and/or relative to similarly situated persons and/or model persons. For example, FIG. 6 illustrates a trait identification of conscientiousness with a trait classification of 100%, indicating relatively high conscientiousness for the patient.

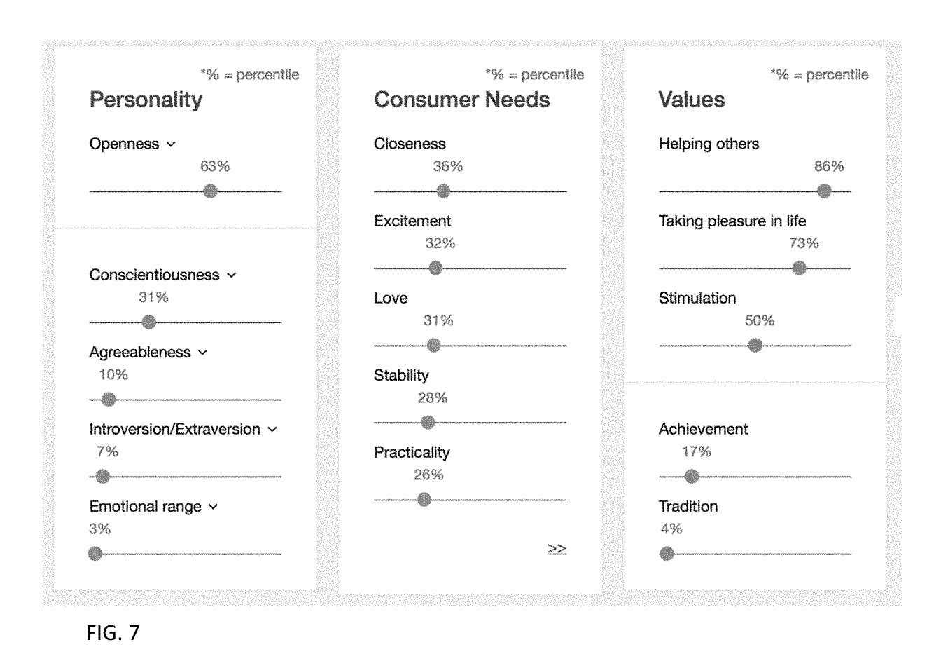

FIG. 7 depicts an aspect of another exemplary personality trait analysis according to one or more embodiments of the present invention. FIG. 7 shows an exemplary personality trait analysis indicative of negative speech, such as speech indicating that a psychologically or medically abnormal personality is present. In some embodiments of the invention, for example, the exemplary personality trait analysis shown in FIG. 7 can indicate a personality shift has occurred or is imminent and can trigger an event notification, such as a positive personality shift notification.

In operation, in an exemplary embodiment of the invention, a schizophrenic patient can have a cell phone microphone that is monitoring his speech patterns and a smart watch monitoring the movements of his right arm. All data collected can go to the cloud. As the patient works in his office, for instance as he participates on a conference call, his speech patterns can be input into the personality extraction module. Accelerometer and gyroscope data is analyzed, for example, in an Internet of Things (IoT) device. Speech patterns and arm movements can be recorded over time to develop a baseline within the personality shift prediction module. Deviations from the baseline can be correlated with data from schizophrenic episodes. For example, quick repetitive arm or leg movements during the call could reflect the onset of a schizophrenic personality change. Multiple repetitions of an out-of-context phrase, such as “I need to go to the hospital” or “call an ambulance,” could verbally indicate the onset of a schizophrenic personality change. Over time, the personality shift prediction module can predict the onset of a schizophrenic episode within a specified confidence level. When a prediction occurs with the specified confidence level, for example, a positive personality shift notification can be sent to family members and caregivers.

Providing positive personality shift notifications to family members and caregivers in real time can allow them to engage and properly direct the actions of the schizophrenic person to prevent unnecessary emergency medical actions and expense.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” are understood to include any integer number greater than or equal to one, i.e. one, two, three, four, etc. The terms “a plurality” are understood to include any integer number greater than or equal to two, i.e. two, three, four, five, etc. The term “connection” can include an indirect “connection” and a direct “connection.”

References in the specification to “one embodiment,” “an embodiment,” “an example embodiment,” etc., indicate that the embodiment described can include a particular feature, structure, or characteristic, but every embodiment may or may not include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may include copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments of the invention, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments of the invention, the practical application or technical improvement over technologies found in the marketplace, or to enable others of ordinary skill in the art to understand the embodiments described herein.

What is claimed is:

1. A computer-implemented method for generating a personality shift determination, the method comprising:
receiving, by a processor, a real-time audio input comprising spoken words of a person;
generating, by the processor, a real-time personality trait identification based at least in part upon the real-time audio input;
generating, by the processor, a current trait classification for the real-time personality trait identification based at least in part upon the real-time audio input and a trait classification model; and
comparing, by the processor, the current trait classification to a historic rate classification; and
generating, by the processor, a personality shift determination based at least in part upon the comparison.

2. The computer implemented method of claim 1 further comprising, outputting a positive personality shift notification based upon a positive personality shift determination.

3. The computer-implemented method of claim 2 further comprising receiving, to the processor, a response to the positive personality shift notification and modifying the trait classification model based at least in part upon the response.

4. The computer implemented method of claim 1 further comprising transcribing the real-time audio input with a speech to text engine.

5. The computer implemented method of claim 1 further comprising receiving, by the processor, real-time movement data.

6. The computer implemented method of claim 1, wherein the historic rate classification comprises a plurality of historic trait classifications for the person.

7. The computer implemented method of claim 1, wherein the historic rate classification comprises a plurality of historic trait classifications for a plurality of similarly situated person.

8. The computer-implemented method of claim 1 further comprising storing the real-time personality trait and the current trait classification in a database in communication with the processor and modifying the trait classification model based at least in part upon the current trait classification.
